(19) United States
(12) Patent Application Publication
(10) Pub. No.: US 2024/0217908 A1
(43) Pub. Date: Jul. 4, 2024

(54) PROCESS FOR MAINTAINING ACTIVITY ON SHUTDOWN OF HYDROFORMYLATION

(71) Applicant: Evonik Oxeno GmbH & Co., KG, Marl (DE)

(72) Inventors: Robert Franke, Marl (DE); Linda Arsenjuk, Dortmund (DE); Florian Ott, Recklinghausen (DE); Jessika Schüller, Gunderath (DE); Peter Kreis, Dortmund (DE); Frank Stenger, Alzenau (DE); Marc Oliver Kristen, Haltern am See (DE)

(73) Assignee: Evonik Oxeno GmbH & Co., KG, Marl (DE)

(21) Appl. No.: 18/539,133

(22) Filed: Dec. 13, 2023

(30) Foreign Application Priority Data
Dec. 19, 2022 (EP) 22214450.3

(51) Int. Cl. C07C 45/50; B01J 21/08; B01J 23/46; B01J 27/224; B01J 31/02; B01J 31/18; B01J 35/50; B01J 37/02

(52) U.S. Cl. CPC C07C 45/505; B01J 21/08; B01J 23/464; B01J 27/224; B01J 31/0237; B01J 31/0271; B01J 31/185; B01J 35/50; B01J 37/0215; B01J 2208/00716; B01J 2231/321; B01J 2531/004

(57) ABSTRACT

A process for hydroformylating short-chain olefins, especially C₂ to C₅ olefins can be performed in at least one reactor in which the catalyst system is in heterogenized form on a support. The support is composed of a porous ceramic material, and the process steps a) to c) are conducted when the process is shut down.

### CROSS-REFERENCE TO RELATED APPLICATIONS

This application claims priority to European Application No. 22214450.3 filed on Dec. 19, 2022, the content of which is hereby incorporated by reference in its entirety.

### BACKGROUND OF THE INVENTION

### Field of the Invention

The project that resulted in this patent application was supported under grant No. 869896 of the of the European Union's Horizon 2020 research and innovation program.

The present invention relates to a process for hydroformylating short-chain olefins, especially C₂ to C₅ olefins, in at least one reactor in which the catalyst system is in heterogenized form on a support composed of a porous ceramic material, and wherein process steps a) to c) as described herein are conducted when the process is shut down.

### Description of Related Art

Hydroformylation is one of the most important reactions in industrial chemistry, having an annual global production capacity of several million tonnes. It involves transforming alkenes (olefins) with a mixture of carbon monoxide and hydrogen (also: synthesis gas or syngas) using a catalyst to aldehydes, which are important and valuable intermediates in the production of chemical bulk products such as alcohols, esters or plasticizers.

Hydroformylation on an industrial scale is carried out exclusively under homogeneous catalysis. The soluble transition metal catalyst systems are typically based on cobalt or rhodium, which is often used together with phosphorus-containing ligands, for example phosphines or phosphites, for the hydroformylation of comparatively short-chain olefins.

There are various problems with the known processes, these problems being linked in particular to the fact that both rhodium and cobalt and compounds thereof are comparatively costly. High energy expenditure and complex process technology are necessary in order to avoid catalyst losses during the hydroformylation process as far as possible, for example through catalyst recycling steps, some of which are very laborious. Moreover, product purification steps are becoming more laborious in order to ensure that as far as possible no catalyst residues remain in the product.

Further problems with the known homogeneously catalysed processes are the stability of the ligands, which have to withstand the conditions of the hydroformylation, such as temperature, pressure, pH etc., and the consumption during the process of the solvent used, which has to be compensated for by replenishment.

In order to get around the abovementioned problems in homogeneously catalysed hydroformylation, hydroformylation processes have been developed in which the catalyst system is heterogenized, especially by immobilization on a support material. The terms “heterogenization” and “immobilization” should accordingly be understood as meaning that the catalyst is immobilized through the formation of a thin liquid film with the aid of an ionic liquid on the surface and/or in the pores of a solid support material and that there is no reaction solution in the conventional sense in which the catalyst is homogeneously dissolved. Hydroformylation processes in which the catalyst is present on a support material in a heterogenized form are disclosed for example in WO 2015/028284 A1, EP 3 632 885 A1, EP 3 744 707 A1, EP 3 632 886 A1 or in EP 3 736 258 A1.

In the industrial implementation of such chemical reactions, it may be necessary time and again to shut down the plant, even for a prolonged period, for example owing to installation or maintenance operations. In the course of such a shutdown, the reaction is first stopped and the reactor is then purged with an inert material, for example nitrogen, in this way, it is possible to discharge the products present in the reactor and preclude further reactions of the products still present. After such a shutdown procedure of the reaction, there can be problems that make it impossible to start up the reaction. The reason for this may be greatly reduced catalyst activity, which would necessitate costly renewal of the catalyst.

### SUMMARY OF THE INVENTION

It was therefore an object of the present invention to provide a process for hydroformylation of olefins that includes steps for shutdown of at least one reactor, but does not have the aforementioned disadvantages. In particular, after the reaction has been restarted, no significantly low catalyst activity should arise, and the activity should instead be very substantially maintained.

This object is achieved in accordance with the embodiment described below in that the at least one reactor is purged with synthesis gas on shutdown and is left to stand in a synthesis gas atmosphere in the subsequent keeping. Preferred configurations are specified in the embodiments

The present invention thus provides a process for hydroformylating C₂ to C₈ olefins in at least one reactor using a heterogenized catalyst system, wherein
a gaseous feed mixture containing the C₂ to C₈ olefins is passed together with synthesis gas in at least one reactor over a support which is arranged in the at least one reactor and is composed of a porous ceramic material on which the catalyst system, which comprises a metal from group 8 or group 9 of the periodic table of the elements, at least one organic phosphorus-containing ligand and a stabilizer, is present in heterogenized form; wherein
the support is a monolith, i.e. a block of a ceramic material, or is in the form of a powder, in the form of a granular material or in the form of shaped bodies and the support consists of a carbidic material, nitridic material, silicidic material or mixtures thereof, and wherein the hydroformylation is conducted at a temperature in the at least one reactor of 65 to 200° C., characterized in that the process comprises the following steps for shutdown and keeping of the at least one reactor:
a) shutting down the feed of gaseous feed mixture while synthesis gas is still being fed into the at least one reactor:
b) lowering the temperature in the at least one reactor to ambient temperature;
c) shutting down the feed of synthesis gas and keeping the at least one reactor under synthesis gas atmosphere until the hydroformylation process is restarted.

The characterizing feature of the present invention is the purging of the at least one reactor with synthesis gas on shutdown of the reaction and keeping it under synthesis gas atmosphere. This can ensure that the process can be run in normal operation again after the reaction has been restarted.

### DETAILED DESCRIPTION OF THE INVENTION

Ambient temperature in the context of the present invention is understood to mean a temperature which is attained without heating or cooling. No more exact definition is possible because the respective prevailing temperature can be significantly different depending, for example, on the time of year.

In the first step a) of the shutdown and keeping steps, the feed of feed mixture is shut down. This stops the reaction. At the same time, the synthesis gas stream is still being supplied to the at least one reactor This allows hydrocarbons still present in the reactor to be purged out. In a preferred embodiment of the present invention, the synthesis gas feed rate is increased when the feed of gaseous feed mixture is shut down in step a). This can ensure that a similar or identical volume flow rate is still being directed through the at least one reactor. In a particularly preferred embodiment, therefore, the feed of gaseous feed mixture is replaced completely by synthesis gas.

In the subsequent step b), the temperature in the at least one reactor is lowered to ambient temperature. This can be effected actively by cooling, in which case the cooling is effected only up to the point where the ambient temperature is attained in the at least one reactor. The cooling can also be effected passively, i.e. without active cooling. In a preferred embodiment of the present invention, the pressure in the at least one reactor is reduced after and/or during the lowering of the temperature in step b). This can remove high boilers possibly still present in the reactor that were not removable by the purging in step a).

In a further preferred embodiment of the present invention, the synthesis gas feed rate can be reduced in step b). This may be necessary under some circumstances in order to be able to achieve a decrease in pressure in the at least one reactor. In addition, it is thus possible to save synthesis gas.

In step c) of the shutdown and keeping steps of the process according to the invention, the feed of synthesis gas is shut down and the at least one reactor is kept in a synthesis gas atmosphere until the hydroformylation is restarted. In a preferred embodiment, before the synthesis gas feed is shut down in step c), the pressure in the at least one reactor can be increased again. If the pressure has been reduced in the preceding step b), the increase in pressure may be necessary.

The at least one reactor can be kept under synthesis gas atmosphere over a particular period of time, for example for days, weeks, months or years. In a preferred embodiment, the at least one reactor is kept under synthesis gas atmosphere for at least 24 hours.

The feed mixture used may be any mixture comprising C₂ to C₈ olefins, preferably C₂ to C₅ olefins, especially ethene, propene, 1-butene, 2-butene, 1-pentene or 2-pentene, as reactants. The amount of olefins in the feed mixtures should understandably be high enough to be able to economically operate a hydroformylation reaction. The feed mixtures that may be employed in the process according to the invention include in particular also technical mixtures from the petrochemical industry, for example raffinate streams (raffinate I, II or III) or crude butane. According to the present invention, crude butane comprises 5% to 40% by weight of butenes, preferably 20% to 40% by weight of butenes (the butenes are composed of 1% to 20% by weight of 1-butene and 80% to 99% by weight of 2-butene), and 60% to 95% by weight of butanes, preferably 60% to 80% by weight of butanes.

The process according to the invention is carried out in at least one reactor in which the hydroformylation according to the invention takes place. The support with the heterogenized catalyst system is arranged in the at least one reactor. In a further embodiment of the present invention, the process can also be carried out in a plurality of reactors, which may be connected in parallel or in series. If two or more reactors are present, these reactors are preferably connected in parallel and are used alternately.

The hydroformylation is preferably carried out under the following conditions: The temperature in the hydroformylation is in the range from 65 to 200° C., preferably 75 to 175° C. and more preferably 85 to 150° C. The temperature may be adjusted by means of a suitable cooling apparatus, for example a cooling jacket. The pressure during the hydroformylation should be at least 1 bar and not greater than 35 bar, preferably not greater than 30 bar, more preferably not greater than 25 bar. The molar ratio between synthesis gas and the feed mixture should be between 6:1 and 1:1, preferably between 5:1 and 3:1. Optionally, the feed mixture can be diluted with inert gas, for example with the alkanes present in technical hydrocarbon streams.

The catalyst system used in the hydroformylation process according to the invention preferably comprises a transition metal from group 8 or 9 of the periodic table of the elements, especially iron, ruthenium, iridium, cobalt or rhodium, further preferably cobalt and rhodium, more preferably rhodium, at least one organic phosphorus-containing ligand and a stabilizer.

The stabilizer is preferably an organic amine compound, more preferably an organic amine compound containing at least one 2,2,6,6-tetramethylpiperidine unit of formula (I):

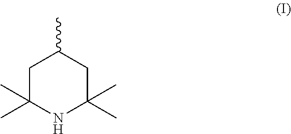

In a particularly preferred embodiment of the present invention, the stabilizer is selected from the group consisting of the compounds of the following formulas (I.1), (I.2), (I.3), (I.4), (I.5), (I.6), (I.7) and (I.8).

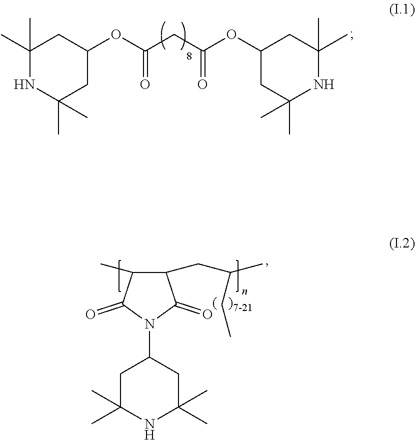

where n is an integer from 1 to 20;

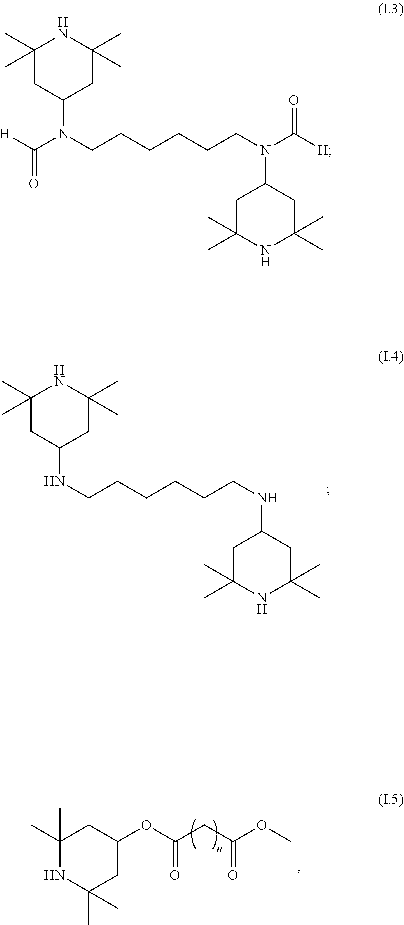

where n is an integer from 1 to 12;

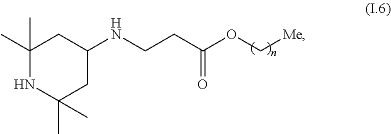

where n is an integer from 1 to 17;

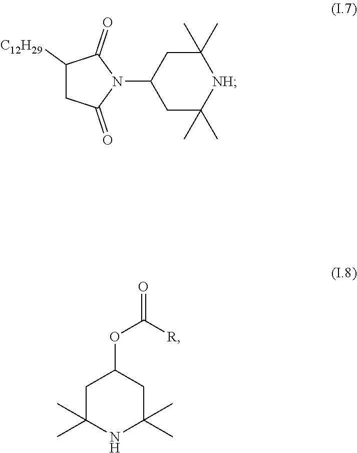

where R is a C₆ to C₂₀ alkyl group.

For all film-forming components, i.e. in this case the stabilizer, the gas solubility for the reactants should be better than the gas solubility of the products. In that way alone, it is possible to achieve partial physical separation between reactant olefins used and product aldehydes formed. In principle, other film-forming substances would also be conceivable for the purpose, but care should be taken to ensure that there is no increased formation of high boilers and/or that the replenishment of the reactant olefins is limited.

The organic phosphorus-containing ligand selected for the catalyst system according to the invention may be any ligand known for hydroformylations. A large number of suitable ligands is known to those skilled in the art from the patent and specialist literature, for example mono- or biphosphite ligands. The organic phosphorus-containing ligand preferably has a biphosphite structure according to the general formula (II)

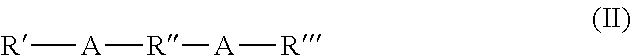

where R′, R″ and R′″ are each organic radicals and the two A are each a bridging —O—P(—O)₂— group, wherein two of the three oxygen atoms —O— are attached respectively to the radical R′ and to the radical R″′, with the proviso that R′ and R″′ are nonidentical, The organic radicals R′, R″ and R′″ preferably do not contain any terminal trialkoxysilane groups.

In a preferred embodiment, R′, R″ and R″′ in the compound of the formula (VI) are preferably selected from substituted or unsubstituted 1.1′-biphenyl, 1,1′-binaphthyl and ortho-phenyl groups, especially from substituted or unsubstituted 1,1′-biphenyl groups, with the proviso that R′ and R″′ are nonidentical. More preferably, the substituted 1,1′-biphenyl groups have an alkyl group and/or an alkoxy group in the 3,3′ and/or 5,5′ positions of the 1,1′-biphenyl base structure, especially a C₁-C₄alkyl group, more preferably a tert-butyl and/or methyl group, and/or preferably a C₁-C₅ alkoxy group, more preferably a methoxy group.

According to the present invention, the abovementioned catalyst system is in heterogenized form on the support composed of a porous ceramic material. In the context of the present invention, the expression “in heterogenized form on a support” is understood to mean that the catalyst system is immobilized on the inner and/or outer surface of the support through the formation of a thin, solid or liquid film with the aid of the stabilizer. The film may also be solid at room temperature and liquid under reaction conditions.

The inner surface of the solid support material comprises more particularly the inner surface area of the pores. The concept of immobilization includes both the situation where the catalyst system and/or the catalytically active species is present in dissolved form in the solid or liquid film and the situation where the stabilizer acts as an adhesion promoter or where the catalyst system is adsorbed on the surface, but not chemically/covalently bonded to the surface.

According to the invention, there is thus no reaction solution in the conventional sense in which the catalyst is homogeneously dissolved; instead, the catalyst system is dispersed on the surface and/or in the pores of the support.

The porous support material is preferably selected from the group consisting of a nitridic ceramic, a carbidic ceramic, a silicidic ceramic and mixtures thereof, for example carbonitridic materials.

The nitridic ceramic is preferably selected from silicon nitride, boron nitride, aluminium nitride and mixtures thereof. The carbidic ceramic is preferably selected from silicon carbide, boron carbide, tungsten carbide or mixtures thereof. Also conceivable are mixtures of carbidic and nitridic ceramic known as carbonitrides. The silicidic ceramic is preferably molybdenum silicide. The support according to the present invention to which the catalyst system is applied preferably consists of a carbidic ceramic, more preferably of silicon carbide.

The support may in the present case be present as a monolith, i.e. a block of a ceramic material, or in the form of a powder, in the form of a granular material or in the form of shaped bodies.

If the support is a monolith, the support consists of a block (a three-dimensional object) of the porous ceramic material. The block may either be in one-piece form or consist of multiple, i.e. at least two, individual parts that may be joined together to form the block and/or may be joined to one another in a fixed or partable manner.

The support composed of the porous ceramic material is preferably a component extending in three dimensions that may in principle be any geometric shape in its cross section, for example round, angular, square or the like. In a preferred embodiment, the component extending in three dimensions that can be used as support has a longitudinal direction (direction of the longest extent) in the main direction of through-flow (direction in which the feed mixture and the synthesis gas flow from the inlet to the outlet of the at least one reactor).

The support monolith of the porous ceramic material thus shaped has at least one continuous channel in the main direction of through-flow. However, the channel(s) may also be configured such that they are not completely continuous but conclude at the opposite end from the inlet of the at least one reactor, i.e. the channel is closed towards this end. The support monolith may also have at least two or more channels. The diameter of the channels may be in the range from 0.25 to 50 mm, preferably in the range from 1 to 30 mm, further preferably in the range from 1.5 to 20 mm and more preferably in the range from 2 to 16 mm. If a plurality of channels is present, the diameters of the channels may be the same or different. The chosen diameter of the channels relative to the diameter or one of the diameters of the overall support should especially be such that the mechanical stability is unimpaired.

In addition, the support monolith of the ceramic material is porous, i e. has pores. The catalyst system according to the invention is more particularly also present in the liquid or solid film in these pores. The pore diameter is preferably in the range from 0.9 nm to 30 μm, preferably in the range from 10 nm to 25 μm and more preferably in the range from 70 nm to 20 μm. Pore diameter can be determined by nitrogen adsorption or mercury porosimetry in accordance with DIN 66133 (1993-06 version).

In a preferred embodiment, the support monolith has at least partly continuous pores that extend from the surface to the channels and/or from one channel to the next channel(s). It is also possible that multiple pores are connected to one another and hence overall form a single continuous pore.

According to the invention, the support may also be present in the form of a powder, in the form of a granular material or in the form of shaped bodies such as pellets, rings, spheres or the like. The median particle diameter (d50) of the support may be from 0.1 mm to 7 mm, preferably 0.3 to 6 mm, more preferably from 0.5 mm to 5 mm. Median particle diameter may be determined by imaging methods, in particular determined by the methods cited in the standards ISO 13322-1 (version: 2004-12-01) and ISO 13322-2 (version: 2006-11-01). The support can be produced in the form of a powder, in the form of a granular material or in the form of shaped bodies by processes known to those skilled in the art. This may for example be done by mechanically comminuting a monolith of the carbidic material, nitridic material, silicidic material or mixtures thereof, for example using a jaw crusher, and adjusting the particle size of the resulting crushed granular material by sieving.

Like the support monolith, the particles of the powder, of the granular material or the shaped bodies composed of the ceramic material are porous, i.e. have pores. The catalyst system according to the invention is more particularly also present in the liquid or solid film in these pores. The pore diameter is preferably in the range from 0.9 nm to 30 μm, preferably in the range from 10 nm to 25 μm and more preferably in the range from 70 nm to 20 μm. Pore diameter can be determined by nitrogen adsorption or mercury porosimetry in accordance with DIN 66133 (1993-06 version).

Whether it be a monolith, powder, granular material or shaped bodies, the support is produced as described below.

It is additionally possible to apply what is called a washcoat, which is composed of the same or a different ceramic material with respect to the ceramic material of the support, preferably silicon oxide, to the provided support composed of the ceramic material, in pulverulent, granular or pellet form. The washcoat itself may be porous or nonporous and is preferably nonporous. The particle size of the washcoat is preferably 5 nm to 3 μm, preferably 7 nm to 700 nm. The washcoat is used to introduce or to generate the desired pore size and/or to increase the surface area of the support. The washcoat can especially be applied by dipping (dip-coating) in a washcoat solution containing the ceramic material of the washcoat, optionally also in the form of a precursor. The amount of washcoat present on the support is ≤20% by weight, preferably ≤15% by weight, more preferably ≤10% by weight, based on the total amount of the support. However, in a preferred embodiment of the present invention the support does not have a washcoat.

The catalyst system is applied to the support with or without washcoat. For this purpose, a catalyst solution is first produced by mixing, especially at room temperature and ambient pressure, the catalyst solution comprising at least one organic phosphorus-containing ligand, at least one metal precursor, for example chlorides, oxides, carboxylates of the respective metal, at least one stabilizer and at least one solvent. It is optionally possible to use an ionic liquid in the production of the catalyst system, but the catalyst solution can also explicitly be prepared without ionic liquid. The catalyst solution should especially be produced in an inert environment, for example in a glovebox. “Inert environment” in this case means an atmosphere that is as far as possible free of water and oxygen.

The solvent may be chosen from all solvent classes (protic, aprotic, polar or nonpolar). A prerequisite for the solvent is the solubility of catalyst system (ligand, metal precursor, stabilizer and optionally the ionic liquid) and preferably also of the high boilers formed in the hydroformylation. Solubility can be increased during the immobilization step by heating.

The solvent is preferably aprotic and polar, for example acetonitrile and ethyl acetate, or else aprotic and nonpolar, for example THE and diethyl ether. Solvents used may also be chlorinated hydrocarbons, for example dichloromethane, or aldehydes.

The catalyst solution thus produced is then contacted with the support (optionally including washcoat), for example by dipping (dip-coating) or by filling a pressurized vessel, for example directly in the at least one reactor (in-situ impregnation). If the catalyst solution is applied outside the reactor, the support must of course be reinstalled in the reactor after the solvent has been removed. Preferably, the catalyst solution is applied to the support with the washcoat directly in the reactor because this can avoid potentially time-consuming installation and deinstallation steps and potential contamination of the catalyst.

The reactor can be filled with the catalyst solution via the normal inlets/outlets, for example by means of a pump. Liquid distributors or nozzles within the reactor can ensure homogeneous distribution of the catalyst liquid, as can pressure drop internals or regulators for the metering rate that are optionally present.

After the catalyst system has been applied, the solvent is removed. This involves firstly discharging the residual catalyst solution via the reactor outlet. Solvent residues remaining in the reactor are then evaporated by adjusting the pressure or increasing the temperature. In another embodiment it is also possible for the pressure to be adjusted in tandem with an increase in temperature. The temperature may be 20 to 150° C., depending on the solvent. The pressure may be adjusted to a high vacuum (10⁻³ to 10⁻⁷ mbar), depending on the solvent, but positive pressures of a few mbar up to several bar are also conceivable, depending on the solvent and temperature.

The stabilizer and the ionic liquid optionally present remain in heterogenized form on the support with the catalyst composed of the transition metal, especially cobalt or rhodium, and the organic phosphorus-containing ligand.

The catalyst system can be applied to the support either directly in the reactor (in situ) or outside the reactor. If the catalyst system is applied outside the reactor, the support should always be transported with exclusion of air, which can be achieved for example with a nitrogen countercurrent. In a preferred embodiment of the present invention, the catalyst system is applied directly in the reactor, i.e. in situ. After the solvent has been removed, the reactor can be used immediately and charged with the feed mixture. This has the advantage that no time-consuming installation and deinstallation steps that would result in a prolonged reactor shutdown are needed. Moreover, this then no longer gives rise to any limitation on the size of the support, in that suitable spaces having inert environments are available in a particular size. The size of the support can be chosen freely according to the reactor design.

Once the catalyst system has been applied to the support and the solvent has been removed, the plant, more particularly the reactor, can be started up, i.e. put into operation, through a two-stage or multistage startup procedure. A suitable startup procedure is described for example in EP 3 632 887.

A gaseous output comprising at least a portion of the product aldehydes formed and at least a portion of the unreacted olefins is preferably withdrawn continuously from the reaction zone in which the hydroformylation according to the invention is carried out. The gaseous output may be subjected to one or more physical separation step(s) in which the gaseous output is separated into at least one phase rich in unreacted olefins and at least one phase rich in product aldehyde.

The physical separation can be carried out by known physical separation methods such as condensation, distillation, centrifugation, nanofiltration or a combination of two or more thereof, preferably condensation or distillation.

In the case of a multistage physical separation, the phase rich in product aldehyde that is formed in the first physical separation can be sent to a second physical separation, especially a downstream removal of aldehyde, in which the product aldehyde is separated from the other substances present in this phase, commonly alkanes and reactant olefins. The phase rich in unreacted olefin can be recycled to the hydroformylation step or, in the case of a multistage configuration, to one of the hydroformylation steps in order to further hydroformylate the olefins present therein to the product aldehyde.

In the physical separation, in addition to the phases mentioned it is also possible to withdraw a purge gas stream having a composition identical or at least similar to the phase rich in unreacted olefin. The purge gas stream can likewise be conveyed to the second physical separation or aldehyde removal in order to remove the product aldehydes present therein and to discharge impurities (for example nitrogen in the synthesis gas) or inert substances (for example alkanes in the feed mixture) from the system. The impurities or inert substances can typically be removed in the second physical separation as volatile substances, for example at the top of a column.

Even without further elaboration it is assumed that those skilled in the art will be able to utilize the description above to the greatest possible extent. The preferred embodiments and examples are therefore to be interpreted merely as a descriptive disclosure which is by no means limiting in any way whatsoever.

The present invention is more particularly elucidated hereinbelow with reference to examples. Alternative embodiments of the present invention are obtainable analogously.

### Example 1

The starting material used for the support was SiC pellets (Sicat SarL). The SiC pellets were introduced into a round reactor sleeve of length 20 cm and having a diameter of one inch (approx. 2.54 cm), with glass beads of similar size introduced above and below the granular material. The SiC pellets were then loaded with a catalyst solution containing Rh(acac)CO)₂, bisphephos (ligand), bis(2,2,6,6-tetramethyl-4-piperidyl)sebacate (stabilizer) and pentanal as solvent. This was done, after first purging the reactor with nitrogen, by introducing the catalyst solution into the reactor under a slight positive pressure. After removal of the solvent from the reactor by discharge and evaporation, the catalyst system heterogenized on the granular support material was used for the hydroformylation.

The feed mixture used for the experiments was a hydrocarbon stream having 15% by weight of butenes and 85% by weight of butanes. For the hydroformylation, the feed mixture was directed into the reactor together with synthesis gas (molar ratio of synthesis gas to feed mixture=3.5.1) at a gas volume flow rate of 390 ml/min. The hydroformylation was carried out at a temperature of 120-130° C. and a pressure of 17 bar During the experiment, the flow of the reactants was stopped for several prolonged periods of time (stoppage time) and, in this stoppage time, the reactor that was still heated to 120° C. was purged with a nitrogen stream at a pressure of 17 bar. Conversion and yield were each determined before and after the stoppage time by means of gas chromatography on the output stream.

TABLE 1
Results for Example 1

| Example 1 | Yield | Conversion |
|---|---|---|
| Before | 24.5% | 45.8% |
| After | 11% | 30.8% |

### Example 2

The starting material used for the support was SiC pellets (Sicat SarL) The SiC pellets were introduced into a round reactor sleeve of length 20 cm and having a diameter of one inch (approx. 2.54 cm), with glass beads of similar size introduced above and below the granular material. The SiC pellets were then loaded with a catalyst solution containing Rh(acac)CO)₂, bisphephos (ligand), bis(2,2,6,6-tetramethyl-4-piperidyl)sebacate (stabilizer) and pentanal as solvent. This was done, after first purging the reactor with nitrogen, by introducing the catalyst solution into the reactor under a slight positive pressure. After removal of the solvent from the reactor by discharge and evaporation, the catalyst system heterogenized on the granular support material was used for the hydroformylation.

The feed mixture used for the experiments was a hydrocarbon stream having 15% by weight of butenes and 85% by weight of butanes. For the hydroformylation, the feed mixture was directed into the reactor together with synthesis gas (molar ratio of synthesis gas to feed mixture=3.5:1) at a gas volume flow rate of 390 ml/min. The hydroformylation was carried out at a temperature of 120-130° C. and a pressure of 17 bar

During the experiment, a shutdown of the reactor was simulated. For this purpose, the flow of the reactants was stopped for prolonged periods of time, and the reactor was cooled down to ambient temperature and started up again after a stoppage time of 23 days. During that period, the reactor was kept under synthesis gas.

In the first step, the flow of the hydrocarbon stream was stopped and replaced at least partially by synthesis gas. After a prolonged purge phase close to the reaction temperature, the pressure in the reactor was lowered in order to drive out further product residues. Thereafter, the system was cooled down to room temperature, and synthesis gas was ultimately injected to keep it at reaction pressure and the system was shut down.

After a stoppage time of 23 days, the system was started up again. For startup, the reactor was first purged again with synthesis gas, synthesis gas was injected to 10 bar, and the reactor was heated to reaction temperature 120° C. The metered addition of the hydrocarbon stream was conducted in multiple stages (as described in EP 3 632 887 A1). Before and after the shutdown and keeping, reactor performance was determined from conversion, yield and selectivity by means of gas chromatography on the output stream.

TABLE 2
Results for Example 2

| | Conversion (total | n/iso | |
|---|---|---|---|
| Example 2 | butenes) | selectivity | Yield |
| Before shutdown | 17% | 96% | 15% |
| After shutdown and | 18% | 96% | 15% |
| keeping | | | |

It is found that, after the shutdown and keeping according to the invention, conversion, selectivity and yield are virtually identical to the values before the shutdown. Catalyst activity has consequently remained virtually constant and not dropped significantly as in Example 1, even though purging in Example 1 was effected solely with nitrogen.

1. A process for hydroformylating C₂ to C₈ olefins in at least one reactor using a heterogenized catalyst system, the process comprising:
passing a gaseous feed mixture containing the C₂ to C₈ olefins together with a synthesis gas in at least one reactor over a support which is arranged in the at least one reactor and comprises a porous ceramic material on which a catalyst system comprising:
a metal from group 8 or group 9 of the periodic table of the elements,
at least one organic phosphorus-containing ligand, and
a stabilizer, is present in heterogenized form; wherein
the support is a monolith in the form of a powder, in the form of a granular material, or in the form of shaped bodies, and wherein the support is selected from the group consisting of a carbidic material, a nitridic material, a silicidic material and mixtures thereof, and wherein the hydroformylation is conducted at a temperature in the at least one reactor of 65 to 200° C., wherein the process comprises the following for shutdown of the at least one reactor:
a) shutting down a feed of the gaseous feed mixture while the synthesis gas is still being fed into the at least one reactor;
b) lowering the temperature in the at least one reactor to ambient temperature;
c) shutting down the feed of the synthesis gas and keeping the at least one reactor under a synthesis gas atmosphere until the hydroformylation process is restarted.

2. The process according to claim 1, wherein the synthesis gas feed rate is increased when the feed of the gaseous feed mixture is shut down in a).

3. The process according to claim 1, wherein a pressure in the at least one reactor is reduced after and/or during the lowering of the temperature in b).

4. The process according to claim 3, wherein the synthesis gas feed rate is already reduced in b).

5. The process according to claim 1, wherein before the synthesis gas feed is shut down in c), a pressure in the reactor is increased again.

6. The process according to claim 1, wherein the at least one organic phosphorus-containing ligand of the hydroformylation catalyst system has the general formula (II)
R′—A—R″—A—R″′ (II)
where R′, R″ and R″′ are each organic radicals, with the proviso that R′ and R′″ are nonidentical, and the two A are each a bridging —O—P(—O)₂— group, wherein two of the three oxygen atoms —O— are attached respectively to the radical R′ and to the radical R″′.

7. The process according to claim 1, wherein the stabilizer is an organic amine compound containing at least one 2,2,6,6-tetramethylpiperidine unit of formula (I):

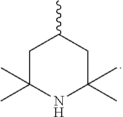

8. The process according to claim 1, wherein the nitridic ceramic is selected from the group consisting of silicon nitride, boron nitride, aluminium nitride and mixtures thereof; the carbidic ceramic is selected from the group consisting of silicon carbide, boron carbide, tungsten carbide and mixtures thereof; and the silicidic ceramic is molybdenum silicide.

9. The process according to claim 8, wherein the support consists of a carbidic ceramic.

10. The process according to claim 9, wherein the support consists of silicon carbide.

11. The process according to claim 1, wherein the hydroformylation is conducted at a temperature in a range from 75 to 175° C.

12. The process according to claim 1, wherein a pressure in the hydroformylation is at least 1 bar and not greater than 35 bar.

13. The process according to claim 1, wherein the catalyst system does not comprise an ionic liquid.

14. The process according to claim 1, wherein C₄ olefins are used in the hydroformylation process.

15. The process according to claim 1, wherein rhodium is used as the metal from group 8 or 9 of the periodic table of the elements.

16. The process according to claim 1, wherein the hydroformylation is conducted at a temperature in a range of from 85 to 150° C.

17. The process according to claim 1, wherein a pressure in the hydroformylation is at least 1 bar and not greater than 25 bar.
